(19) United States
(12) Patent Application Publication
(10) Pub. No.: US 2020/0210852 A1
(43) Pub. Date: Jul. 2, 2020

(54) TRANSCRIPTOME DECONVOLUTION OF METASTATIC TISSUE SAMPLES

(71) Applicant: TEMPUS LABS, INC., Chicago, IL (US)

(72) Inventors: Catherine Igartua, Chicago, IL (US); Kaanan Shah, Chicago, IL (US); Mathew Barber, Chicago, IL (US)

(21) Appl. No.: 16/732,229

(22) Filed: Dec. 31, 2019

Related U.S. Application Data
(60) Provisional application No. 62/944,995, filed on Dec. 6, 2019.
(60) Provisional application No. 62/924,054, filed on Oct. 21, 2019.
(60) Provisional application No. 62/786,756, filed on Dec. 31, 2018.

(51) Int. Cl. G06N 3/12; G16B 40/20; G16B 40/30; G16B 30/10; G16H 10/40

(52) U.S. Cl. CPC G06N 3/123; G16B 40/20; G16H 10/40; G16B 30/10; G16B 40/30

(57) ABSTRACT

A platform for transcriptome deconvolution of gene expression data is provided and may be used in assessing metastatic cancer samples. The deconvolution is performed using an unsupervised clustering technique, such as grade of membership, that allows for samples to be assigned to multiple clusters during a training process. A deconvolution gene expression model is generated as a result and is used for accurate assess of metastases in subsequent samples.

### CROSS-REFERENCE TO RELATED APPLICATIONS

This patent application claims the benefit of U.S. Prov. App. No. 62/786,756 filed Dec. 31, 2018; U.S. Prov. App. No. 62/924,054 filed Oct. 21, 2019; and U.S. Prov. App. No. 62/944,995 filed Dec. 6, 2019. All of the aforementioned applications are incorporated in their entirety by reference herein. U.S. application Ser. No. 16/533,676 and Int. Patent App. No. PCT/US19/45368, titled “Multi-Modal Approach to Predicting Immune Infiltration Based on Integrated RNA Expression and Imaging Features” (filed Aug. 6, 2019), also are incorporated in their entirety by reference herein, and particularly with respect to disclosure relating to systems and methods for deconvolution (e.g., use of deconvolution to determine amounts of cell populations present in a specimen).

### FIELD OF THE INVENTION

The present disclosure relates to the transcriptome analysis of mixed cell type populations and, more particularly, to techniques for the deconvolution of RNA transcript sequences quantified in metastatic tumor tissues.

### BACKGROUND

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Solid tumors are heterogeneous mixtures of cell populations composed of tumor cells, nearby stromal and normal epithelial cells, immune and vascular cells. Transcriptome profiling of tumor samples by standard RNA (ribonucleic acid) sequencing methods measures the average gene expression of the cell types present in the sample at the time of sampling, the samples generally including both tumor (target) and non-tumor (non-target) cells. The expression profile is largely shaped by the sample's tumor architecture. Tumor purity, i.e., the proportion of cancerous cells in the sample, can directly influence the sequencing results, genomic interpretation, and any consequent proposed associations with clinical outcomes. Put another way, as clinical tumor samples comprise a mixed population of cells, many of which are non-tumor cells, a resulting gene expression profile may not concisely reveal clinically relevant associations. The dependence on tumor purity and the challenge it poses to genomic interpretation is most pronounced in metastatic cancers, where the tumor and the non-cancerous background tissue can have different gene expression profiles, due to the tumor originating in a tissue that is distinct from the background tissue where the tumor has metastasized. In other words, RNA expression from normal adjacent cells to the tumor could increase or wash out the relevant expression signal for a given gene and result in the erroneous interpretation of over or under expression and subsequent treatment recommendations.

Motivated to understand tumor heterogeneity and to model transcription profiles in cancer, a few computational approaches have been developed to estimate cell type specific expression profiles in tumor cells. These methods have mainly focused on the disassociation of immune cells from tumor samples and require known expression references from well characterized cell-type specific genes, or transcriptomes from purified cell populations. In spite of existing methods, the deconvolution of tumor gene expression from the surveyed mixture of cell populations containing unwanted normal cells in the collected tissue remains a challenging task. There is a need for improved transcriptome deconvolution techniques.

### SUMMARY OF THE INVENTION

The present application presents novel techniques for transcriptome deconvolution and in particular techniques for using transcriptome deconvolution to assess metastatic cancer samples. In an example, the present techniques are used to examine metastatic tumors from multiple cancer types.

In one example, the present techniques include quantifying the proportion of a sample that is normal cells, compared to the proportion that is tumor or cancer cells. In one example, the samples are 4,754 cancer and liver normal samples. The present techniques may include the quantification of transcriptome signatures to estimate the proportion of non-tumor cells in mixture samples. Certain techniques include adjusting gene expression profiles in a regression-based approach against reference samples, based on the proportion of the sample that is estimated to be healthy tissue. This adjustment of gene expression profiles in the tumor may be utilized to accurately model tumor features in a sample such as, for instance, the prediction of cancer type, detection of over and under expression of gene and pathway activity, characterization of cancer molecular subtypes/networks, biomarker discovery, and clinical associations, among others, to inform better response or resistance to treatment.

In some examples, the present techniques may quantify metastatic samples. In an example, the proportion of liver in each sample in a set of 4,754 cancer and liver normal samples is quantified and then used to train a non-negative least squares model to estimate liver proportion in mixture samples. The liver normal samples may be non-tumorous liver tissue. The information derived from the samples may be RNA expression data, such as measured RNA levels. The mixture samples may be metastatic tissue samples, including tumor and background non-tumor cancer site cells, such as normal tissue adjacent to the metastasized tumor, which may be included as part of a biopsy or surgical removal. Estimated liver proportions across mixture samples may then be utilized to adjust gene expression profiles in a regression-based approach. The techniques, while described as used for liver samples and liver cancer, can be extended to other types of tissue samples or cancers, whether those samples are metastatic or not. Examples of normal tissue include but are not limited to liver, brain, lung, lymph node, bone marrow, bone, abdomen, and pleura, or any portion of the human body. The mixture samples may further include immune cells (including dendritic cells, lymphocytes, macrophages, etc.).

The cancer in some aspects is one selected from the group consisting of acute lymphocytic cancer, acute myeloid leukemia, alveolar rhabdomyosarcoma, bone cancer, brain cancer, breast cancer (e.g., triple negative breast cancer), cancer of the anus, anal canal, or anorectum, cancer of the eye, cancer of the intrahepatic bile duct, cancer of the joints, cancer of the head or neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, gastrointestinal cancer (e.g., gastrointestinal carcinoid tumor), glioblastoma, Hodgkin lymphoma, hypopharynx cancer, hematological malignancy, kidney cancer, larynx cancer, liver cancer, lung cancer (e.g., non-small cell lung cancer (NSCLC), small cell lung cancer (SCLC), bronchioloalveolar carcinoma), malignant mesothelioma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, and urinary bladder cancer. The listing of cancers herein is not intended to be exhaustive in scope, other cancers may be considered as well.

In an example, a computer-implemented method comprises: performing clustering on RNA expression data corresponding to a plurality of samples, where each sample is assigned to at least one of a plurality of clusters; generating a deconvoluted RNA expression data model comprising at least one cluster identified as corresponding to biological indication of one or more pathologies; receiving additional RNA expression data of a sample of tumor tissue; deconvoluting the additional RNA expression data based in part on the deconvoluted RNA expression data model; and classifying the sample of tumor tissue as the biological indication of one or more pathologies.

In some examples, clustering on the RNA expression data is performed using a grade of membership clustering operation. In some examples, the grade of membership clustering operation is performed iteratively until the at least one cluster corresponding to the biological indication is identified. In other examples, clustering on the RNA expression data is performed using a non-negative matrix factorization operation.

In some examples, the generated deconvoluted RNA expression data model comprises a first dimension reflecting a number of samples and a second dimension reflecting a number of genes in the RNA expression data.

In accordance with another example, a computer-implemented method comprises: receiving RNA expression data for a tissue sample of interest; comparing the received RNA expression data to a deconvoluted RNA expression model comprising at least one cluster identified as corresponding to biological indication of one or more pathologies; and determining a pathology type for the tissue sample of interest based on the comparison.

In some examples, comparing the received RNA expression data to the deconvoluted RNA expression model includes deconvoluting the received RNA expression data.

In accordance with another example, a computer-implemented method comprises: receiving RNA expression data for a tissue sample of interest; comparing the received RNA expression data to a deconvoluted RNA expression model comprising at least one cluster identified as corresponding to biological indication of one or more cell types; and determining one or more cell types present in the tissue sample of interest based on the comparison.

In some examples, the one or more cell types comprises cell populations, collections of cells, populations of cells, stem cells, and/or organoids.

In accordance with another example, a method, comprises: receiving RNA expression information of a sample of tumor tissue; generating a deconvolution of the RNA expression information; and determining a biological indication of the tumor tissue based in part on the deconvolution.

In some examples, the biological indication is a cancer type. In some examples, the biological indication of the tumor tissue is a metastatic cancer.

In some examples, determining the biological indication of the tumor tissue includes: generating enriched gene expressions; and classifying the enriched gene expressions in a biological indication data model. In some examples, generating enriched gene expressions includes: receiving membership associations to each cluster of the plurality of clusters; and scaling the RNA expression information for one or more genes based in part on the corresponding membership associations to each cluster.

In some examples, deconvolution is performed with a supervised machine learning model, a semi-supervised machine learning model, or an unsupervised machine learning model.

In some examples, the RNA expression data is raw. In some examples, the RNA expression data is normalized RNA expression data.

The techniques, while described as used to deconvolute RNA expression data, can be extended to deconvolute DNA read count data, including for example, DNA read counts measured by a genetic sequence analyzer.

### BRIEF DESCRIPTION OF THE DRAWINGS

This patent or application file contains at least one drawing executed in color. Copies of this patent or patent application publication with color drawing(s) will be provided by the United States Patent and Trademark Office upon request and payment of the necessary fee.

The figures described below depict various aspects of the system and methods disclosed herein. It should be understood that each figure depicts an example of aspects of the present systems and methods.

FIG. 1 is a schematic illustration of an example computer processing system having a deconvolution framework for performing deconvolution on RNA expression data, in accordance with an example.

FIG. 2 is a block diagram of an example process for generating deconvoluted RNA expression data from normalized, metastatic sample RNA expression data as may be performed by the system of FIG. 1, in accordance with an example.

FIG. 3 is a block diagram of an example implementation of the deconvoluted RNA expression data generating process of FIG. 2, in accordance with an example.

FIG. 4 is a block diagram of an example implementation of the development of a deconvolution regression model of block 312, in accordance with an example.

FIG. 5 is a plot of principal component analysis (PCA) of gene expression profiles of reference tissue samples.

FIG. 6 is a plot of proportions for a Grade of Membership (GoM) model with K=15 clusters, in an example implementation of the deconvolution framework of FIG. 1. The K=15 clusters were fit to 4,754 samples from 22 cancers and normal liver. Each sample is represented as a horizontal bar plot of membership proportions to the 15 clusters. Samples are ordered by cancer/tissue type and sorted by cluster proportions of K=1 within each group.

FIG. 7 illustrates a distribution of the GoM cluster K=5 by cancer and tissue type for the 4,754 samples example of FIG. 6. As shown, normal liver GTEx and TCGA lihc samples have the highest proportion of the K=5 latent factor, while TCGA primary cancers have the lowest.

FIG. 8 illustrates results of a leave one out validation of the deconvolution frame, specifically a liver deconvolution model generated by the framework, in accordance with an example. A non-negative least squares (NNLS) model of tumor estimates is shown to be highly correlated to the GoM proportion (r=0.98) of the present techniques.

FIGS. 9 and 10 are plots of principal component analysis of a pancreatic cohort before (FIG. 9) and after (FIG. 10) deconvolution of liver metastases, in accordance with an example. The PCA analysis included 65 pancreatic samples (labelled by their background tissue site) along with TCGA primary liver (lihc) and pancreatic (paad) samples and GTEx normal liver samples. After deconvolution (FIG. 10), liver metastatic samples form a group with all other pancreatic cancer samples.

FIGS. 11A and 11B are plots of PCA analysis of breast and liver in silico mixtures and deconvoluted modelling results, for two different samples. As shown, after deconvolution is applied to the liver mixture RNA expression data, the proper grouping of liver samples occurs.

FIG. 12 is a summary of expression call results in original RNA expression data and in deconvoluted RNA expression data, in accordance with an example. Values are the proportion of samples with calls in each of the groups among the cancers where that gene had at least one sample called.

### DETAILED DESCRIPTION

As used herein, the following terms have the associated meanings.

“Biological validation” refers to the comparison of a set of identified genes that are correlated with a cluster and genes represented in RNA expression profiles known or likely to be associated with a subset of tissues, including a portion of a tissue sample, a type of cell that may be in a tissue sample, or single cells within a tissue sample and may determine a correlation between the known RNA expression profile genes and the genes correlated with a cluster, associating the cluster with the expression profile of that subset of tissue.

“Cluster” refers to a set of genes whose expression levels are correlated with a percentage of the variance seen among multiple samples in an RNA expression dataset. The cluster may be said to be driven by this set of genes, where “driven” is a term for describing that the expression levels of the genes in this set explain a percentage of the variance. The expression levels of the genes in this set may have patterns that are consistently associated with the variance. For example, the expression level of a given gene in the set may be higher or lower in samples having one or more characteristics in common, or the expression levels of two or more genes may be directly or inversely correlated with each other in samples having one or more characteristics in common. Sample characteristics may include the collection site of the sample, the type of tissue or combination of tissue types contained in the sample, etc.

“Bioinformatics pipeline” means a series of processing stages of a pipeline to instantiate bioinformatics reporting regarding next-generation sequencing results of a patient's tumor or normal tissue or bodily fluids to extract and report on variants present in the patient's genome.

“Deconvolution” refers to a process of resolving expression data from a mixed population of cell types to identify expression profiles of one or more constituent cell types, for example using algorithm processes.

“Expression level” means the number of copies of an RNA or protein molecule generated by a gene or other genetic locus, which may be defined by a chromosomal location or other genetic mapping indicator.

“Gene product” means a molecule (including a protein or RNA molecule) generated by the manipulation (including transcription) of the gene or other genetic locus, which may be defined by a chromosomal location or other genetic mapping indicator.

“Genetic analyzer” means a device, system, and/or methods for determining the characteristics (including sequences) of nucleic acid molecules (including DNA, RNA, etc.) present in biological specimens (including tumors, biopsies, tumor organoids, blood samples, saliva samples, or other tissues or fluids).

“Genetic profile” means a combination of one or more variants, RNA transcriptomes, or other informative genetic characteristics determined for a patient from next-generation sequencing.

“Genetic sequence” means a recordation of a series of nucleotides present in a patient's RNA or DNA as determined from sequencing the patient's tissue or fluids.

“Metastatic sample” refers to a sample of a tumor that arose from an organ different from the organ from which the sample was taken.

“Mixed purity metastatic cancer sample” refers to a metastatic sample that includes adjacent non-cancerous tissue.

“Normal sample” refers to a sample of non-tumor tissue.

“Primary sample” refers to a sample of a tumor that arose from the same organ from which the sample was taken.

“Reads” refers to the number of times that a sequence from a sample was detected by a sequencer.

“RNA read count” means the read counts of RNA or cDNA generated from a genetic analyzer.

“Sequencing depth” refers to the total number of repeated reads per nucleotide in a sample.

“Sequencing probe” means a collection of chemicals which attach to a locus of a chromosome based on the expected sequence of nucleotides at the RNA or DNA present at that locus.

“Targeted Panel” means a combination of probes for next-generation sequencing of a patient's biological specimens (including tumors, biopsies, tumor organoids, blood samples, saliva samples, or other tissues or fluids) which are selected to map one or more loci on one or more chromosomes.

“Variant” means a difference in a genetic sequence or genetic profile when compared to a reference genetic sequence or expected genetic profile.

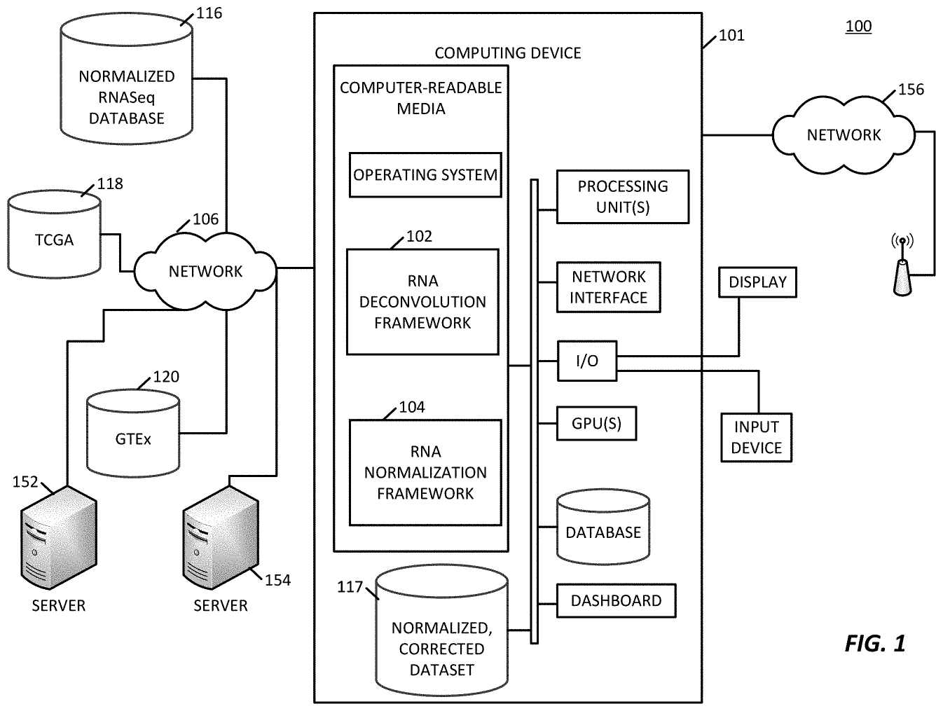

A system for performing deconvolution on gene expression data and developing a deconvolution model for gene expression analysis is shown in FIG. 1. The system 100 includes computing device 101 for implementing the techniques herein. As illustrated, the computing device 101 includes a deconvolution framework 102 and a RNA normalization framework 104, both of which may be implemented on one or more processing units, e.g., Central Processing Units (CPUs), and/or on one or more or Graphical Processing Units (GPUs), including clusters of CPUs and/or GPUs. Features and functions described for the deconvolution framework 102 and the normalization framework 104 may be stored on and implemented from one or more non-transitory computer-readable media of the computing device 101. The computer-readable media may include, for example, an operating system and the frameworks 102 and 104. More generally, the computer-readable media may store batch normalization process instructions for the framework 104 and deconvolution process instructions for the framework 102, for implementing the techniques herein. The computing device 101 may be a distributed computing system, such as an Amazon Web Services cloud computing solution.

The computing device 101 includes a network interface communicatively coupled to network 106, for communicating to and/or from a portable personal computer, smart phone, electronic document, tablet, and/or desktop personal computer, or other computing devices. The computing device further includes an I/O interface connected to devices, such as digital displays, user input devices, etc.

The functions of the frameworks 102 and 104 may be implemented across distributed computing devices 152, 154, etc. connected to one another through a communication link. In other examples, functionality of the system 100 may be distributed across any number of devices, including the portable personal computer, smart phone, electronic document, tablet, and desktop personal computer devices shown. The computing device 101 may be communicatively coupled to the network 106 and another network 156. The networks 106/156 may be public networks such as the Internet, a private network such as that of a research institution or a corporation, or any combination thereof. Networks can include, local area network (LAN), wide area network (WAN), cellular, satellite, or other network infrastructure, whether wireless or wired. The networks can utilize communications protocols, including packet-based and/or datagram-based protocols such as Internet protocol (IP), transmission control protocol (TCP), user datagram protocol (UDP), or other types of protocols. Moreover, the networks can include a number of devices that facilitate network communications and/or form a hardware basis for the networks, such as switches, routers, gateways, access points (such as a wireless access point as shown), firewalls, base stations, repeaters, backbone devices, etc.

The computer-readable media may include executable computer-readable code stored thereon for programming a computer (e.g., comprising a processor(s) and GPU(s)) to the techniques herein. Examples of such computer-readable storage media include a hard disk, a CD-ROM, digital versatile disks (DVDs), an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory) and a Flash memory. More generally, the processing units of the computing device 200 may represent a CPU-type processing unit, a GPU-type processing unit, a field-programmable gate array (FPGA), another class of digital signal processor (DSP), or other hardware logic components that can be driven by a CPU.

The computing device 101 is coupled to receive gene expression count data from a database, such as a gene expression dataset 116. In one example, gene expression data may be normalized counts or raw RNA expression counts, which report the number of times that a particular gene's RNA is detected in a sample by a sequence analyzer or another device for detecting genetic sequences. The computing device 101 may be coupled to receive gene expression data from a multitude of different, external sources through the communication network 106. The computing device 101, for example, may be coupled to a health care provider, a research institution, lab, hospital, physician group, etc., that makes available stored gene expression data in the form of an RNA sequencing dataset. Example external gene expression datasets include the Cancer Genome Atlas (TCGA) dataset 118 and the Genotype-Tissue Expression (GTEx) dataset 120, both examples of established gene expression datasets that can be normalized by the normalization framework 104 and incorporated into an already-normalized database of gene expression data, such as the dataset 116. The gene expression dataset 116 may be a normalized dataset. Methods of normalizing gene expression data are disclosed in U.S. patent application Ser. No. 16/581,706, filed Sep. 24, 2019, which is incorporated by reference in its entirety. A gene expression dataset may be obtained, e.g., from a network accessible external database or from an internal database. The gene expression dataset may contain RNA seq data. A gene information table containing information such as gene name and starting and ending points (to determine gene length) and gene content (“GC”) may be accessed and the resulting information used to determine sample regions for analyzing the gene expression dataset 116.

In an example, additional normalizations may be performed. For instance, a GC content normalization may be performed using a first full quantile normalization process, such as a quantile normalization process like that of the R packages EDASeq and DESeq normalization processes (Bioconductor, Roswell Park Comprehensive Cancer Center, Buffalo, N.Y., available at https://bioconductor.org/packages/release/bioc/html/DESeq.html). The GC content for the sampled data may then be normalized for the gene expression dataset. Subsequently, a second, full quantile normalization may be performed on the gene lengths in the sample data. To correct for sequencing depth, a third normalization process may be used that allows for correction for overall differences in sequencing depth across samples, without being overly influenced by outlier gene expression values in any given sample. For example, a global reference may be determined by calculating a geometric mean of expressions for each gene across all samples. A size factor may be used to adjust the sample to match the global reference. A sample's expression values may be compared to a global reference geometric mean, creating a set of expression ratios for each gene (i.e., sample expression to global reference expression). The size factor is determined as the median value of these calculated ratios. The sample is then adjusted by the single size factor correction in order to match to the global reference, e.g., by dividing gene expression value for each gene by the sample's size factor. The entire GC normalized, gene length normalized, and sequence depth corrected RNA seq data may be stored as normalized RNA Seq data. A correction process may then be performed on the normalized RNA seq data, by sampling the RNA Seq data numerous times, and performing statistical mapping or applying a statistical transformation model, such as a linear transformation model, for each gene. Corresponding intercept and beta values may be determined from the linear transformation model and used as correction factors for the RNA seq data.

In some examples, the normalization framework 104, to incorporate multiple datasets, includes a gene expression batch normalization process that adjusts for known biases within the dataset including, but not limited to, GC content, gene length, and sequencing depth. The normalization framework 104 includes a gene expression correction process. The normalization framework 104 may generate one or more correction factors, which are applied by the normalization framework 104 to convert new gene expression datasets, such as datasets 118 and 120, into a normalized dataset. Applying these correction factors, the normalization framework 104 is able to normalize, correct, and convert the new gene expression dataset 116 for integration into an existing normalized, corrected gene expression dataset 117, as shown. Known biases include, for example, two unnormalized datasets may not be compared directly if the datasets were acquired by different sequencing protocols. Additionally, some characteristics of a genetic sequence in a sample may change the likelihood that the sequencer will detect that sequence. The distribution of nucleotides of a genetic sequence (percentage of guanosine (G) or cytosine (C), versus adenine (A) or thymine (T)) can influence the likelihood of sequences being amplified and detected by a sequencer. Similarly, decreased gene sequence length and lower sequencing depth decreases the likelihood of gene-level sequence read detection and quantification. In these cases, the normalization process multiplies the reads by a correction factor that adjusts the number of reads to better reflect the actual number of molecular copies of those sequences in the sample.

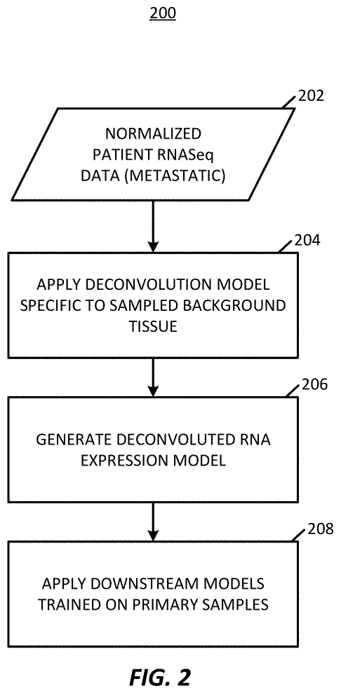

The deconvolution framework 102 may be configured to receive normalized gene expression data and modify such data using a clustering process to optimize the number of clusters, K, such that one or more gene expression clusters associated with one or more cell types of interest are detected. Subsequent analysis of the gene expression clusters may determine cancer-specific cluster types within such data. The deconvolution framework is discussed with more detail with respect to FIG. 2 below.

Deconvoluted gene expression data may be used in downstream gene expression data analyses and may yield more accurate results than analyzing mixed sample gene expression data. For example, analyses of the mixed sample gene expression data may return results that reflect the background tissue instead of the cancer tissue in the mixed sample. Examples of downstream gene expression data analyses include determining which genes are overexpressed or underexpressed, determining consensus molecular subtypes, predicting a cancer type present in the sample (especially for tumors of unknown origin), detecting infiltrating lymphocytes, determining which cellular activity pathways are dysregulated, discovering biomarkers, matching therapies or clinical trials based on the results of any of these downstream analyses, and designing clinical trials or organoid experiments based on the results of any of these downstream analyses.

In one example, predicting the cancer type present in a metastatic sample biopsied from the liver by analyzing mixed sample gene expression data may result in a prediction that liver cancer is present in the sample, when it is actually metastatic breast cancer.

In another example, the deconvolution framework 102 receives DNA read count data associated with a mixed sample and deconvolutes the DNA read count data to provide deconvoluted DNA read count data for one of the tissue types within the mixed sample. This deconvoluted DNA read count data may be used in downstream DNA data analyses and may yield more accurate results than analyzing mixed sample DNA read count data. Examples of downstream DNA data analyses include detecting variants, calculating variant allele fraction, detecting copy number variation, detecting homologous recombination deficiency, discovering biomarkers, matching therapies or clinical trials based on the results of any of these downstream analyses, and designing clinical trials or organoid experiments based on the results of any of these downstream analyses.

FIG. 2 illustrates a process 200 that may be executed by the system 100, and in particular the deconvolution framework 102, to perform an exemplary deconvolution on RNA expression data. At a block 202, the system 100 receives normalized RNA expression data, e.g., from the normalized RNA sequence database 116. In some examples, the system 100 is configured to generate the normalized RNA expression data, e.g., as described in reference to the normalization framework 104. The RNA expression data may contain data for various tissue samples, including cancer tissue samples and normal tissue samples. The RNA expression data, as described in various examples herein, may include metastatic tissue samples, which contain a mixture of cancer and normal tissue. The samples may be from any tissue type, including by way of example, liver tissue, breast tissue, pancreatic tissue, colon tissue, bone marrow, lymph node tissue, skin, kidney tissue, lung tissue, bladder tissue, bone, prostate tissue, ovarian tissue, muscle tissue, intestinal tissue, nerve tissue, testicular tissue, thyroid tissue, brain tissue, and fluid samples (e.g., saliva, blood, etc.). The sample may also be an organoid, for example, an organoid derived from a tumor and grown in vitro.

At a block 204, the deconvolution framework 102 analyzes the normalized RNA expression data and applies a deconvolution model to remove expression data from cell populations that are not cell types of interest (e.g. tumor or other types of cancer tissue). In some examples, the block 204 implements the deconvolution model using machine learning algorithms such as unsupervised or supervised clustering techniques to examine gene expression data to quantify the level of tumor versus normal cell populations present in the data. The block 204 may apply any number of machine learning algorithms, such as, for example, anomaly detection, artificial neural networks, expectation-maximization, singular value decomposition, etc. In some examples, the block 204 may apply machine learning techniques. Other example machine learning techniques that may be used in place of clustering include support vector machine learning, decision tree learning, associated rule learning, Bayesian techniques, and rule-based machine learning.

In some examples, and as discussed further herein, the block 204 analyzes multiple samples of tissue applying the deconvolution model to identify one or more correlated clusters of RNA expression data and the genes corresponding to those clusters for identifying tissue and cancer types in subsequent RNA expression data. After completing the clustering process, the block 204 generates a deconvoluted RNA expression model that is stored (at block 206) for use as a trained model to examine subsequently received RNA expression data, such as RNA expression data generated from a tissue sample from a patient with cancer. For example, the deconvoluted RNA expression model may include regressed out clusters corresponding to latent factors, e.g., clusters of gene expression data corresponding to particular cancer types or cell populations with similar expression profiles, especially clusters that correspond to a cell population that has an effect on the mixed sample RNA expression data that is subtracted from the expression data (for example, regressed out) to generate a deconvoluted RNA expression model. These deconvoluted RNA expression models, as shown by examples below, are able to exhibit overexpressed genes and underexpressed genes different from those of normal or mixed, convoluted RNA expression data and that more accurately predict cancer type based on the list of those overexpressed and underexpressed genes. The generated trained deconvoluted models may then be applied to subsequent RNA expression data, at a block 208.

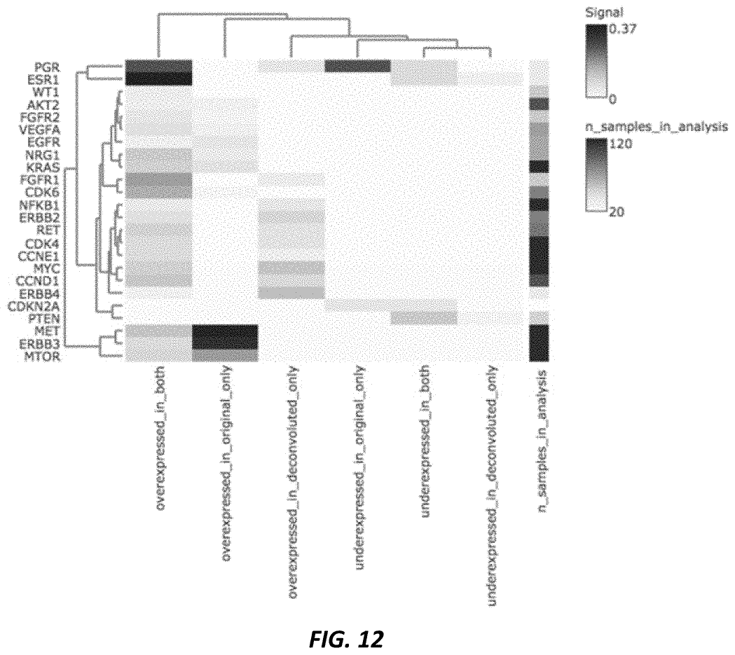

RNA expression data examined by the deconvoluted RNA expression model may be used to determine which genes, or networks of related genes, have expression levels that differ between tumor and normal tissue. Exemplary differences in expression levels in deconvoluted versus convoluted RNA expression data are depicted in FIG. 12. In various aspects, comparing tumor expression levels with normal tissue levels permits biomarker discovery, by determining which genes or gene networks have a higher or lower expression level in tumor tissue than normal tissue that may be adjusted or targeted by treatment. Such a comparison permits predicting the type of cancer or the origin of the cancer, associating mutations with gene expression patterns, and associating tumor gene expression profiles with a list of cancer treatments that may predict response for a patient with that profile.

As part of deconvolution, the number of genes or networks of related genes in the datasets to be analyzed may be in the thousands or tens of thousands.

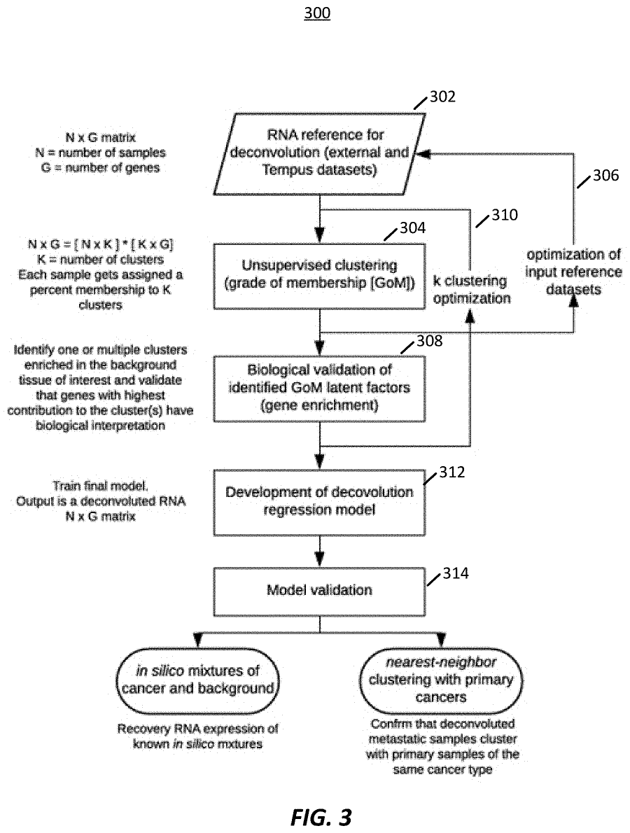

FIG. 3 illustrates a detailed example implementation of a process 300 for generating a deconvolution RNA expression data model, as may be performed by the system 100 to implement the process 200. In an initial training mode, reference RNA expression data is received at a block 302. This reference RNA expression data may be normalized RNA expression data from external and/or internal datasets. External datasets may include RNA sequence data from gene expression databases, such as the TCGA database 118 and the GTEx database 120, that may not be normalized to a database, such as the normalized database 116. The RNA expression data may be configured in a N×G matrix, where N is the number of samples and G is the number of genes. An expression level value associated with a gene may represent the combined amount of all transcripts that can be a product of that gene (for example, splice variants and/or isoforms), or an expression level may be a single transcript or subset of transcripts associated with that gene. In one example, there are approximately 19,000 genes and approximately 160,000 unique transcripts associated with the human genome. In some examples, the RNA expression data includes data from normal samples, primary samples (such as breast tumor from breast tissue), and metastases samples (such as breast tumor from liver tissue). In some examples, if primary samples are not available or not available in large numbers, non-cancerous samples from the tissue matching the cancer type of the primary sample may be used instead of or in addition to the primary samples (for example, non-cancerous breast tissue instead of primary breast cancer samples).

A block 304 receives RNA expression data from block 302 and analyzes the RNA expression data with a clustering algorithm executed by the processing device. In the illustrated example, the clustering algorithm may apply a grade of membership (GoM) model, which is a mixture model that allows sampled RNA expression data to have partial memberships in multiple clusters, as the clustering algorithm executes. For example, in each cycle, each sample, N, within the RNA expression data may be assigned a percentage membership in each of the K number of clusters. This computing device continues the process via a processing loop 306 until the samples are clustered across each of the RNA expression datasets. The clustering algorithm may be implemented using the CountClust algorithm (Bioconductor, Roswell Park Comprehensive Cancer Center, Buffalo, N.Y., available at https://bioconductor.org/packages/CountClust/). For instance, grade of membership may be implemented in CountClust using a fit on normalized, log₁₀ gene expression counts for K=10, 12, 14, 16, and 24 clusters. Gene enrichment, which identifies if any of the members of a list of genes or proteins has a class of genes or proteins that is represented more than statistically expected, may be calculated on the top 1,000 driving genes reported for each cluster using the process instructions for the goseq R package (Bioconductor, Roswell Park Comprehensive Cancer Center, Buffalo, N.Y., available at https://bioconductor.org/packages/release/bioc/html/goseq.html). In other examples, alternative algorithms may be used to determine the optimal number of clusters. In another example, an alternative clustering algorithm may be run, including, but not limited to, Non-negative matrix factorization (NMF). In various embodiments, the clustering is unsupervised and does not require the use of reference gene expression profiles generated from pure tissue or cell type samples for deconvolution.

The number of clusters may be predetermined or dynamically set by the block 304. For example, the number of clusters may be dependent upon the type of tissue being sampled in the RNA expression data, the type and heterogeneity of cancer types or cell populations to be examined, or the sample size distribution of the reference samples and the type of sequencing technology. An exemplary training dataset may include RNA expression data from tissue normal samples, primary samples, and metastatic samples. An alternative training set may also include labels, annotations, or classifications identifying each of the samples as the respective type of tissue, in addition to other biological indicators (such as cancer site, metastasis, diagnosis, etc.) or pathology classifications (such as diagnosis, heterogeneity, carcinoma, sarcoma, etc.).

A machine learning algorithm (MLA) or a neural network (NN) may be trained from the training data set. MLAs include supervised algorithms (such as algorithms where the features/classifications in the data set are annotated) using linear regression, logistic regression, decision trees, classification and regression trees, Naíve Bayes, nearest neighbor clustering; unsupervised algorithms (such as algorithms where no features/classification in the data set are annotated) using Apriori, means clustering, principal component analysis, random forest, adaptive boosting; and semi-supervised algorithms (such as algorithms where certain features/classifications in the data set are annotated) using generative approach (such as mixture of Gaussian distributions, mixture of multinomial distributions, hidden Markov models), low density separation, graph-based approaches (such as mincut, harmonic function, manifold regularization), heuristic approaches, or support vector machines. NNs include conditional random fields, convolutional neural networks, attention based neural networks, long short term memory networks, or other neural models where the training data set includes a plurality of samples and RNA expression data for each sample. While MLA and neural networks identify distinct approaches to machine learning, the terms may be used interchangeably herein. Thus, a mention of MLA may include a corresponding NN or a mention of NN may include a corresponding MLA.

Training may include identifying common expression characteristics shared across RNA gene expressions in tissue normal samples, primary samples, and metastatic samples, such that the MLA may predict the ratio of a metastases tumor from the background tissue and identify which portion of an input RNA expression set may be attributed to the tumor and which portion may be attributed to the background tissue. Common expression characteristics may include which genes are expected to be overexpressed, expressed, and/or underexpressed for each type of tissue and/or tumor and may be identified for each k cluster as the corresponding genes. In one example, for training a supervised MLA, the annotations provided for each sample would be a full transcriptome gene expression dataset, cancer type, tissue site, and background tissue percentage. In one example, liver normal would be labeled 100% background tissue while primary cancers would be labeled 0% background tissue.

With the samples clustered with partial memberships using the process of block 304, at a block 308, the computer device may perform an optional biological validation of identified grade of membership latent factors. This process is also referred to as gene enrichment in the present example, which is the analysis of a list of genes or proteins to identify any classes of genes or proteins that are represented by members of the list at a rate that is higher than statistically expected. In an example implementation, one or more clusters enriched in genes known to be associated with the background tissue of interest are identified by the computing device. The block 308 then determines which genes have the highest contribution to these clusters, and the block 308 validates that these genes have biological interpretation. For the validation, for example, the computing device may compare the identified genes against a pre-existing database of genes associated with particular biological processes that are to be examined and are known to be relevant in the cell population of interest. For instance, the cell population of interest may be liver cells, breast cancer cells in a tumor, etc. In this way, the biological validation may determine which cell type is associated with each cluster by analyzing the genes that are over or under expressed in the cluster and matching it to a list of genes known to be over or under expressed in a cell type. For example, if a cluster has high gene expression for genes associated with liver tissue (including CYP genes, etc.) then this biological validation step may determine that the cluster represents liver cells.

In one embodiment, biological validation may include comparing each sample's estimated membership percent in a given cluster with that sample's tumor purity estimate (or 1-tumor purity) to determine whether the cluster is likely to represent the primary cancer cells (or background tissue cells) in the sample. Proportion estimates for other cell types that are known for a mixed sample may be used in a similar fashion to associate a cluster with that cell type. In various examples, tumor purity of a mixed sample may be determined by visual analysis of a histopathology slide or by bioinformatic analysis of DNA data associated with the sample.

The processes of blocks 304 and 308 may be performed using a feedback 310 until cluster optimization is complete. Clustering may be applied multiple times to yield a varying number of clusters, K, and the membership percentages of all samples of each type of tissue in each cluster may be analyzed. An optimal number of K clusters may be selected such that the membership sum of one or multiple clusters has i) high estimated proportion in reference samples with the cell population of interest (such as liver normal and liver cancers), ii) low proportion in other cell types (such as non-liver primary cancers) and iii) the strongest significant enrichment of relevant biological pathways (such as metabolic processes for identification of liver background).

With the biological validation completed from the block 308, at a block 312, the deconvolution framework 102 develops a deconvolution regression model of RNA expression data. The deconvolution regression model may be developed by calculating the contribution of one or more clusters to gene expression levels and removing those contributions from a sample's gene expression data. In one example, the effect of a specific membership percentage in a given cluster on the expression level of a given gene may be calculated by using a regression of RNA expression data derived from multiple samples (plotted as the sample's membership percentage in the cluster on the x-axis and the sample's expression level for that gene on the y-axis). The block 312 stores a deconvoluted RNA matrix of N×G values as the regression model, or a first matrix of N×K values with a second matrix of K×G values, for example. In this example, N represents each sample, K represents each cluster, and G represents each gene. There may be a row or column in a matrix for each sample, cluster, and/or gene.

Because the number of clusters may be optimized in block 308, the systems and methods disclosed herein do not require a limit to the number of cells present in the sample and may be used to generate a deconvoluted transcriptome for each of the cell types, for any number of cell types. In one example, a mixed sample may contain metastatic cancer tissue, immune cells, and background tissue from the biopsy collection site. Any portion of the human body may be a background tissue type in the mixed sample, including, but not limited to liver tissue, brain tissue, lung tissue, lymph nodes, bone marrow, bone, pleura, abdomen, etc. The immune cells may include multiple cell types (including lymphocytes, macrophages, dendritic cells, etc.), and the background tissue may have multiple cell types (including stroma, epithelium, and cells specific to an organ, for example, hepatocytes in the liver). The mixed sample may be an organoid, including multiple types of tumor cells (for example, clones) and/or multiple immune cell types. In one example, each cell type expected in the mixed sample is assigned to at least one of the clusters defined by the clustering algorithm during the biological validation step. For example, the clustering algorithm identifies K number of clusters and then the biological validation step determines a biological representation of each of those clusters by identifying clusters enriched with genes that are representative of those cell types (for example, immune cells, hepatocytes, and endothelial cells). Then, at the block 312, a regression model having separate terms for each of those estimated proportions is built, accounting for more than one cluster. In one example, each cluster may be interpreted as more than one cell population.

The deconvoluted RNA matrix may be validated at a block 314, which may perform an in silico validation (i.e., validation performed on a computer) for example by using in silico mixtures of cancers and background RNA expression data. The validation analyzes whether the deconvoluted RNA matrix properly identifies, from the samples, RNA expressions of known in silico mixtures. In another example, the block 314 performs validation using a machine learning technique, such as analyzing the RNA expression data sets before and after deconvolution using a grouping analysis known as nearest neighbor clustering and comparing the results of the grouping analysis. This validation may be applied to confirm that relevant samples of the deconvoluted RNA matrix will form a group with primary samples of the same cancer type when sorted by a grouping technique.

In one example, these validations may be used to determine if there is a lower minimum tumor purity that serves as a limit of detection. For example, if the deconvoluted RNA matrix of in silico samples having a cancer proportion below a threshold do not resemble the cancer RNA expression data used to make the in silico sample, that threshold may be a limit of detection. In another example, if the deconvoluted RNA matrix of samples having a tumor purity below a threshold do not form a group with primary samples of the same cancer type when sorted by a grouping technique, that threshold may be a limit of detection.

In another example, the validation may further include an analysis of the distribution of the number of latent factor reads (for example, background tissue reads) subtracted (for example, regressed out) from the sample's data set during deconvolution, across the population of samples. A histogram may be used to visualize the number of samples (y-axis) having a particular number of sequencing reads subtracted from each sample's data set (x-axis) to determine whether the distribution of subtracted reads is heterogenous. If the distribution is not heterogenous, for example, if the majority of samples have either very few reads subtracted and/or a large number of reads subtracted, this may indicate that the algorithm is finding a local minimum or local maximum because not all of the data sets used to train the deconvolution model are comparable. The data sets may not be comparable because of batch effects, differences in normalization, or other causes of differences between genetic data sets. This incompatibility within the training data set may need to be corrected (for example, by normalizing the training data with normalization framework 104) prior to optimizing the deconvolution model.

Returning to FIG. 2, application of the MLA described above with respect to FIG. 3 at block 204 of FIG. 2 may include receiving RNA expression data of a metastatic tumor in a patient. For example, a patient may be diagnosed with breast cancer which has metastasized to additional locations in the patient's body and a breast cancer tumor may now be present in the patient's liver. The tissue sample processed by a genetic sequence analyzer may have included both the breast tumor tissue and healthy liver tissue, so the convoluted, mixed tissue sample that is sequenced will include expression results from both tissues. The gene expression levels of both tissues will contribute to the measured gene expression levels of the total, mixed sample.

An exemplary model, trained as described above with respect to FIG. 3, may process the received RNA expression data to identify the membership of each cluster of the model (i.e., in a k=15 model where k is the number of clusters, each sample receives 15 different membership classifications, one associated with each cluster). In an unsupervised MLA, an exemplary cluster may not be assigned to any particular cancer site with tumor, cancer site without tumor, or metastases tumor, as an unsupervised algorithm clusters based off of similar features without regarding particularly the classification of each sample. Therefore, it may not be easy to identify which features correspond to which type of sample. In an unsupervised approach, only the genes whose expression levels are predicted to have been affected by the sample's membership in one or more of the clusters are identified and then the expression levels of those genes are adjusted in post processing (i.e., using variate/multivariate regression) to counteract the effects of the sample's percentage of membership in any of the clusters.

For a particular sample, the MLA result may identify a percentage of membership in each cluster (e.g., 15% k₁, 65% k₉, 20% k₁₃). Post processing of the grade of membership output may include a multivariate regression which will accommodate for the influence of each cluster, for example k₁, k₉, and k₁₃ in the RNA expression data. In an exemplary embodiment, a linear regression based on the expression levels of one gene in all of the training samples that had membership in one of the respective clusters may, for each gene, be used to calculate a regressed gene expression level. For example, if a cluster was derived from 1000 samples, each sample may be plotted as a data point with the grade of membership percentage in that cluster on the x-axis and the expression level of a given gene in the sample on the y-axis and the equation of a regression line may be calculated to approximate the plotted data points. Using the equation of the regression line, it is possible to replace x with the membership percentage of the newest sample, and calculate the y, which is the expression level of the gene that is explained by that percentage of membership in that cluster. In one example, to remove the effect of that cluster, the calculated expression level y may be subtracted from the total gene expression level measured in the mixture sample for that gene. In another example, the expression level of each gene associated with that cluster may be scaled to increase or decrease the gene expression level measured in the mixture sample based on where the gene's expression falls in relation to the average at that membership percentage on the linear regression plot.

By calculating each cluster's effect on the expression levels of all genes associated with the cluster, these factors may be regressed out (i.e., by summing the initial RNA gene expression level measured in the mixture sample with the additive inverse of each cluster's effect) and the resulting deconvoluted RNA expression data may be evaluated for biomarkers or other biological indications. In a supervised or semi-supervised MLA, an exemplary cluster will be assigned to one or more types of samples (particular cancer site with tumor, cancer site without tumor, or metastatic tumor). For example, k₅ may be assigned to breast tumor, k₆ may be assigned to tumorous breast tissue metastasized in the liver, and k₇ may be assigned to non-tumor breast tissue. Furthermore, the initial training dataset may include a table of the N samples which identifies the corresponding type of sample. Therefore the output from the MLA processing may identify a percentage of membership within each cluster as well as a prediction of the type of sample. Post-processing for semi-supervised and supervised MLA may be performed in the same manner as the unsupervised MLA described above.

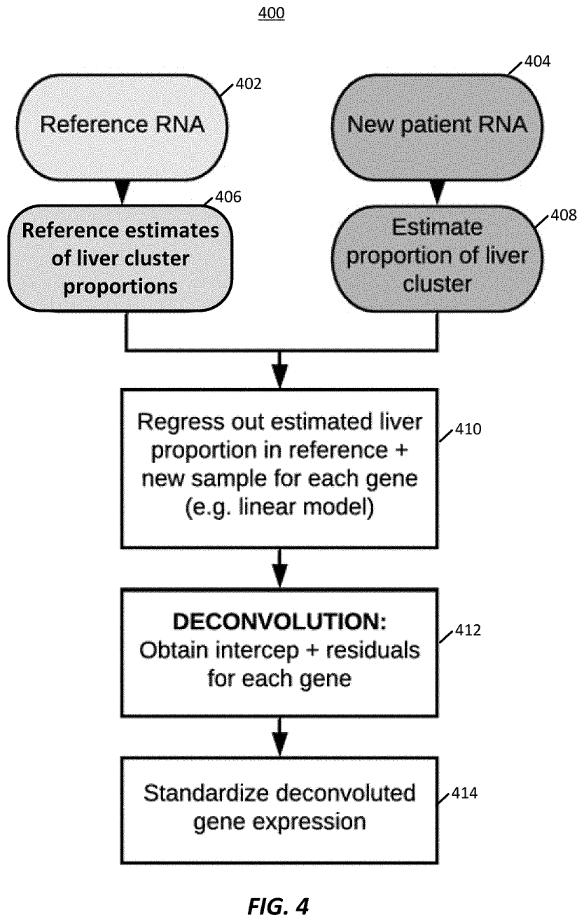

FIG. 4 is a block diagram of an example implementation 400 of the development of a deconvolution regression model of block 312, in accordance with an example.

RNA data sets from reference databases 402/404 (for example, GTEx and TCGA databases) and from patients or organoids are received. Each RNA data set 402/404 is associated with a biological sample, and an estimate of the proportion of background tissue (for example, liver) present in the sample is determined at processes 406 and 408, respectively. The proportion of background tissue is equal to 1-tumor purity. Each RNA data set 402/404 contains expression levels, each of which is associated with a gene.

For each gene, at a process 410, a linear model is generated to correlate the proportion of background tissue present in a sample with the expression level of the gene associated with that sample.

At a process 412, corresponding intercept and beta (for example, residual) values may be determined from the linear model and used as correction factors to generate a standardized deconvolution model. At a process 414, the intercept and beta values may be used to adjust each RNA data set that was received, or any additional RNA data set, to remove any gene expression level correlated with the proportion of background tissue associated with that RNA data set.

### EXAMPLES

We now describe an example implementation of the processes of FIGS. 2, 3, and 4, in particular as applied to an example analysis of liver metastatic samples.

Initially, we compiled a reference dataset comprising 238 sequenced liver metastatic samples (Tempus Labs, Inc., Chicago, Ill.), 120 metastatic samples as part of a Met500 project, 3,508 primary samples from The Cancer Genome Atlas (TCGA) selected from among 22 cancers in the metastatic liver samples, and 136 normal liver samples from the Genotype-Tissue Expression project (GTEx), Table 1 (4,754 samples in total).

In this example, samples were collected as part of GTEx, TCGA, Met500 projects or clinical samples (Tempus Labs, Inc., Chicago, Ill.). To minimize possible batch effects, raw data from GTEx and TCGA databases were downloaded in bam file format and processed through the same RNA-seq pipeline for sequence alignment and normalization. Met500 and clinical samples underwent a RNA-seq library preparation approach that included a transcription capture step and was optimized for formalin-fixed paraffin-embedded (FFPE) samples. To account for differences in library preparation methods across studies, we calculated per gene sizing factors on log₁₀ normalized counts from 500 subsamples of 1,000 TCGA and clinical samples from a group of 9,295 TCGA samples and 3,903 clinical samples. Sizing factors were applied to TCGA and GTEx samples to ensure genes had equivalent mean and variances across studies.

TABLE 1
Sample composition for samples included
in the grade of membership reference.
Selected TCGA samples include all cancers present in the
liver metastatic cancer set, which comprises the 238
sequenced liver metastatic samples (Tempus Labs, Inc.,
Chicago, IL) and 120 metastatic samples from the Met500 project.

| | Tempus | Met500 | GTEx | TCGA |
|---|---|---|---|---|
| Liver metastases | 238 | 120 | | |
| Liver normal | | | 136 | |
| Primary cancers | 752 | | | 3,508 |
| Total | 990 | 120 | 136 | 3,508 |

GTEx: Genotype-Tissue Expression; TCGA: The Cancer Genome Atlas.

The most abundant cancers within the liver metastases were breast (23.5%), pancreatic (19.8%) and colon (17.3%) cancers (Table 2).

TABLE 2
Distribution of cancer and tissue types by study in the reference set.

| | Liver | | | | |
|---|---|---|---|---|---|
| | metastases | | | | |
| | (Tempus and | TCGA | Tempus | GTEx | |
| Cancer | Met500) | primary | primary | liver | Total |
| Adrenocortical carcinoma (acc) | 3 | 45 | 0 | 0 | 48 |
| Bladder Urothelial Carcinoma (blca) | 8 | 202 | 19 | 0 | 229 |
| Breast invasive carcinoma (brca) | 84 | 529 | 169 | 0 | 782 |
| Cholangiocarcinoma (chol) | 17 | 14 | 0 | 0 | 31 |
| Colon adenocarcinoma (coad) | 62 | 137 | 80 | 0 | 279 |
| Diffuse large B-cell lymphoma(difflc) | 1 | 21 | 1 | 0 | 23 |
| Esophageal carcinoma (esca) | 1 | 79 | 16 | 0 | 96 |
| Head and Neck squamous cell carcinoma (hnsc) | 11 | 259 | 1 | 0 | 271 |
| Kidney chromophobe (kich) | 1 | 32 | 0 | 0 | 33 |
| Kidney renal clear cell carcinoma (kirc) | 6 | 267 | 64 | 0 | 337 |
| Liver hepatocellular carcinoma (lihc) | 5 | 179 | 0 | 0 | 184 |
| Liver (normal) | 0 | 0 | 0 | 136 | 136 |
| Lung adenocarcinoma (lnad) | 5 | 249 | 94 | 0 | 348 |
| Lung squamous cell carcinoma (lnsc) | 3 | 239 | 33 | 0 | 275 |
| Ovarian serous cystadenocarcinoma (ov) | 5 | 180 | 52 | 0 | 237 |
| Pancreatic adenocarcinoma (paad) | 71 | 79 | 59 | 0 | 209 |
| Pheochromocystoma and Paragangiloma (pcpg) | 20 | 88 | 22 | 0 | 130 |
| Prostate adenocarcinoma (prad) | 31 | 246 | 68 | 0 | 345 |
| Sarcoma (sarc) | 11 | 118 | 2 | 0 | 131 |
| Skin cutaneous melanoma (skcm) | 2 | 223 | 8 | 0 | 233 |
| Stomach adenocarcinoma (stad) | 8 | 168 | 17 | 0 | 193 |
| Thyoma (thym) | 2 | 70 | 14 | 0 | 86 |
| Uterine corpus endometrial (ucen) | 1 | 84 | 33 | 0 | 118 |
| Total | 358 | 3,508 | 752 | 136 | 4,754 |

In this example, a validation step was performed that uses principal component analysis (PCA) to assess groupings based on RNA gene expression profiles among the primary cancer samples, healthy tissue samples, and the deconvoluted metastatic samples. PCA, performed by computing devices such as that of FIG. 1, is a dimension reduction technique for comparing data sets from multiple samples or a single data set containing multiple samples, especially where each sample may be associated with multiple values, such as an expression level value for each expressed gene for tens of thousands of expressed genes or more. PCA may be used on all expressed genes to determine which genes in conjunction have the greatest variance in expression levels among samples.

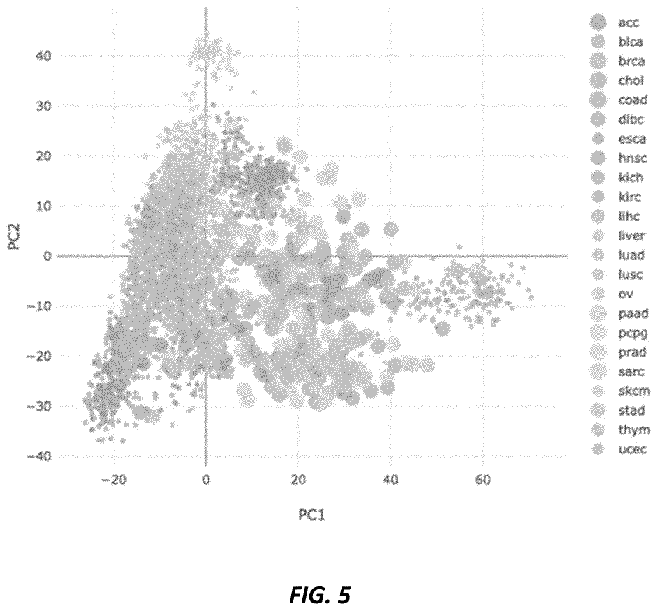

The principal components may be sorted according to the largest percent of variance explained by the contributions of those genes to demonstrate the greatest differences among samples, and the principal component that makes the largest contribution to variance may be designated principal component 1 (PC1). The principal component that makes the second largest contribution to variance (after regressing out the contribution of PC1) may be designated principal component 2 (PC2). The samples may be spatially arranged according to the extent of contribution principal components that contribute the largest percentage of the variance in the dataset. In the example shown in FIG. 5 generated by the computing device, the expression levels of the group of genes represented by PC1 distinguishes samples with a low proportion of liver cells (in the example, primary non-liver cancers) from samples with a high proportion of liver cells (in the example, liver cancer and healthy liver samples). The expression levels of the group of genes represented by PC2 distinguishes samples based on differences caused by primary cancer types. As expected, liver specific cancers and liver tissue do not contain this type of variance and there is not a large degree of separation along the y-axis for these groups.

The groups of sample data can be visually represented in a chart such as the one shown in FIG. 5. Samples are colored by their tissue or origin. As shown, PC1 explained 10.5% of the variance and separated the TCGA liver hepatocellular carcinoma (lihc) and GTEx normal liver from the other non-liver primary cancers. Rather than forming a group with their cancer type of origin, in this unsupervised grouping example, principal component analysis grouped the liver metastatic samples together as a continuum between the TCGA cancers and liver normal (GTEx) and cancer samples (lihc TCGA). Metastatic liver samples (meaning, tumor cells from another organ which are found in the liver) are represented with larger circles and formed groups away from their respective TCGA primary cancers. As shown in FIG. 5, small circles to the left of liver metastases represent non-liver primary cancers, while liver primary cancers and liver normal samples are represented by small circles that group to the right of the metastases. This variation in expression separating metastatic liver samples from primary samples is attributable to the expression of the normal background liver tissue in the sample. As shown, rather than grouping with their cancer type of origin, liver metastatic samples grouped together as a continuum between the TCGA cancers, on the left, and both liver normal (GTEx liver) and liver cancer samples (TCGA liver hepatocellular carcinoma (lihc)) on the right.

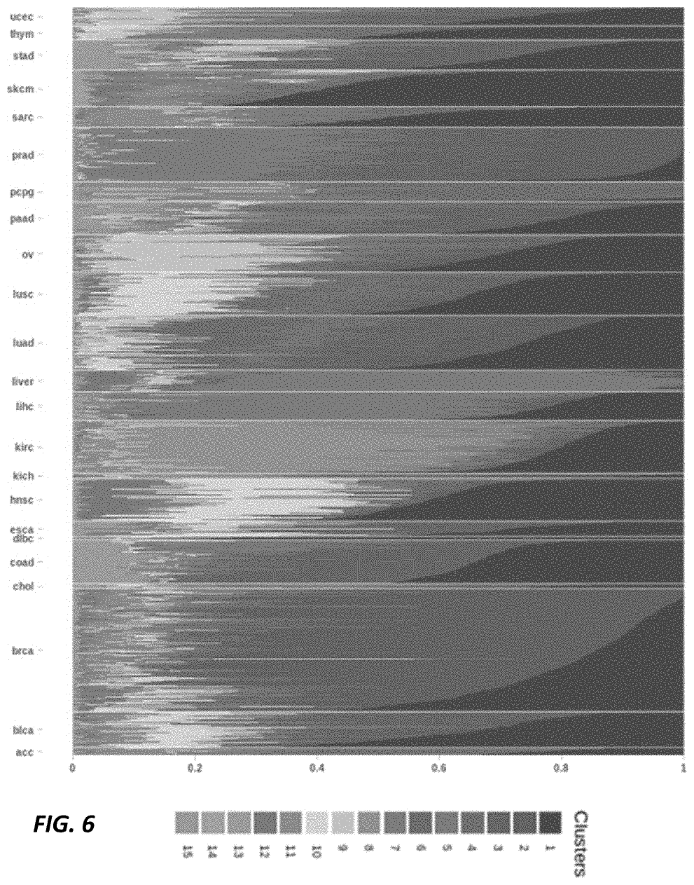

Aiming to characterize the cell populations present in the samples, the CountClust algorithm was used as an exemplary clustering algorithm to fit a grade of membership model (GoM) with 15 clusters (K=15). The clustering shown in FIG. 6 illustrates the 15 clusters and the top 1,000 genes driving each of the clusters as determined using the CountClust algorithm GoM model. In FIG. 6, the labels on the left indicate cancer types or liver normal tissue, each row represents a single sample of the cancer type indicated on the left, and each color represents a cluster associated with a portion of that sample (see legend at bottom of FIG. 6). The length of each color in each row relative to the length of the entire row represents the percentage of that row's sample that is associated with the cluster of that color.

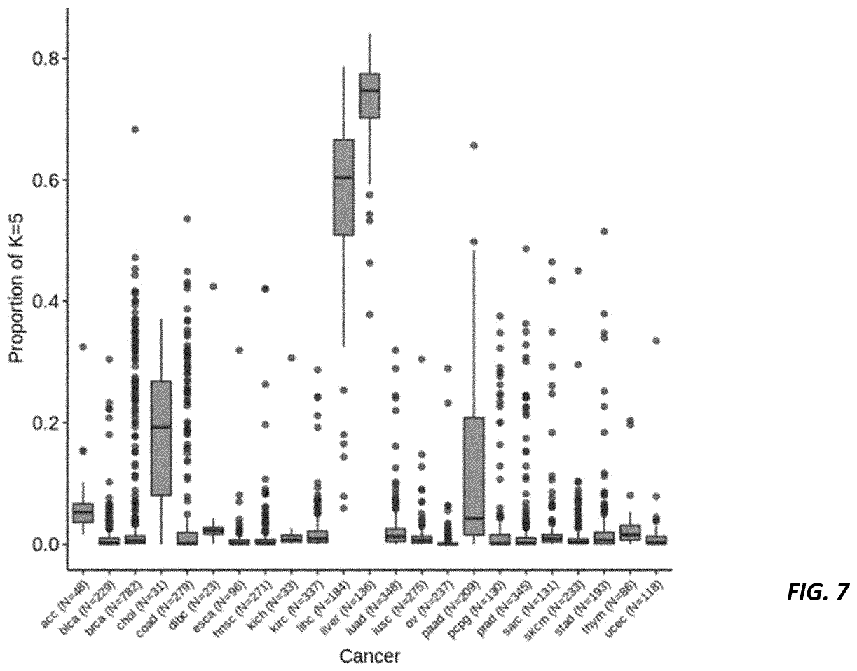

A preferred cluster size, meaning the number of clusters, may be K=15. Cluster size was selected such that a single cluster results in high estimated proportions in GTEx liver and TCGA lihc samples and low in other TCGA cancer samples, as shown in FIG. 6 as the olive green colored band that indicates cluster number 5 (see legend). We identified one cluster (the fifth cluster, or k=5, colored in olive green) where TCGA lihc, chol and GTEx liver samples had high membership proportions (average 0.608, 0.192, and 0.730, respectively) and all other, non-liver TCGA primary cancers resulted in low proportions (0.011). Metastatic liver samples had a range of intermediate membership values for the 5th cluster (0.230), as shown in FIG. 7, which illustrates the distribution of the fifth GoM cluster by cancer type for all 4,754 samples. FIG. 7 is a box plot representation of the membership values of the samples within each cancer or tissue type labeled along the x-axis of the plot, with dots representing the outliers in each category. The metastatic samples with low tumor purity and high background tissue are likely to be outliers, with higher proportions of the fifth cluster. Liver metastatic samples from Met500 and from Tempus Labs, Inc. had intermediate estimated proportions for this cluster. Primary Pancreatic Ductal Adenocarcinoma (paad) and Cholangiocarcinoma bile duct cancer (chol) contain tissues that have gene expression profiles that are similar to liver tissue, which accounts for the high estimated proportions of the fifth cluster in these cancer samples.

As an optional validation, to assign biological relevance to the particular fifth cluster, a gene enrichment method (available at http://geneontology.org/) was configured to select the top 1,000 genes influencing the fifth cluster and perform gene enrichment analyses for Gene Ontology (GO) biological processes. This gene enrichment analysis identified 582 biological processes that were significantly enriched after Bonferroni correction, meaning that 582 biological processes were disproportionately associated with the genes whose expression was most consistently correlated with the fifth cluster. Metabolic processes were among the most enriched, with the most significant being GO:0019752—carboxylic acid metabolic process (203 out of 1,002 genes; p=3.61×10⁻⁸⁵). Given this result, we consider the fifth cluster to be a liver specific latent factor and an approximation to the proportion of liver background tissue present in each sample and comparable across samples.

The determination of the fifth cluster as a liver specific latent factor was validated against tumor purity data. Tumor purity estimates for 140 samples were available from DNA sequencing of the same tumor sample and from pathology estimates from separate samples. This allowed us to assess the correlation between the fifth GoM cluster proportion and these tumor purity estimates and found correlations of −0.33. The result is trained and validated identification of a cluster for use in predicting cancer and liver percentages. In the example of process 300, this procedure may repeat through feedback 310 until all clusters are examined and validated.

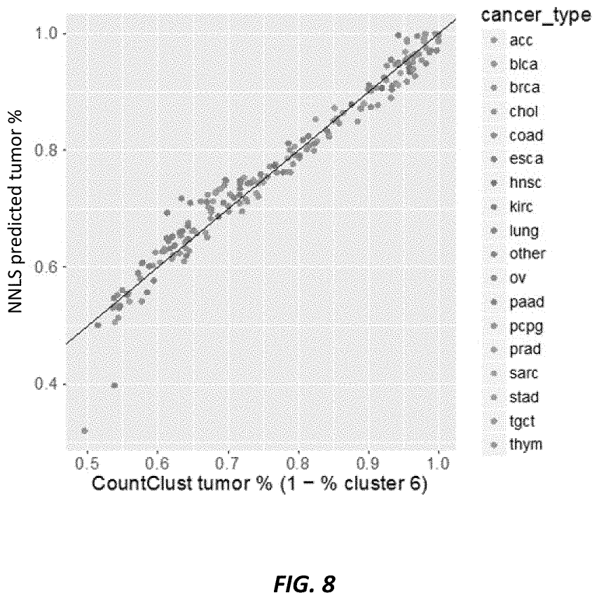

In one example, the present techniques may implement a non-negative least squares (NNLS) model, to predict tumor and liver percentages trained on the GoM proportions of the fifth cluster and gene expression profiles from 358 liver metastatic samples. We selected 500 genes with the lowest sum of square error (SSE) in a leave-one-out validation approach applied to all genes. We then validated the selected gene list in a second leave-one-out step that resulted in a correlation of r=0.98 between predicted liver proportions and equivalent performance across cancer types, as shown in FIG. 8.

In one example, a customized non-negative least squares algorithm estimates cell proportions within a sample and projects them to a probability simplex such that all estimates are non-negative and sum up to one. Optimization of the convex function was done iteratively such that the sum of squares error (SSE) between the model parameters and the sample estimates have a difference of less than 10⁻⁷ between the two most recent runs. To select a set of genes with the highest predictive power in the final non-negative least squares model, we performed a leave-one-out NNLS approach using gene expression of 19,147 genes across 358 liver metastatic samples. We used the GoM proportion of the fifth cluster (liver) and 1 minus this proportion as predictors. The technique may be used to predict origin of cancer. We selected 500 genes with the lowest SSE among the models for our final model implementation. While the number of selected genes is somewhat arbitrary, we selected 500 genes from among a series of gene sets (100, 250, 500) such that GO enrichment associations reached the highest significance.

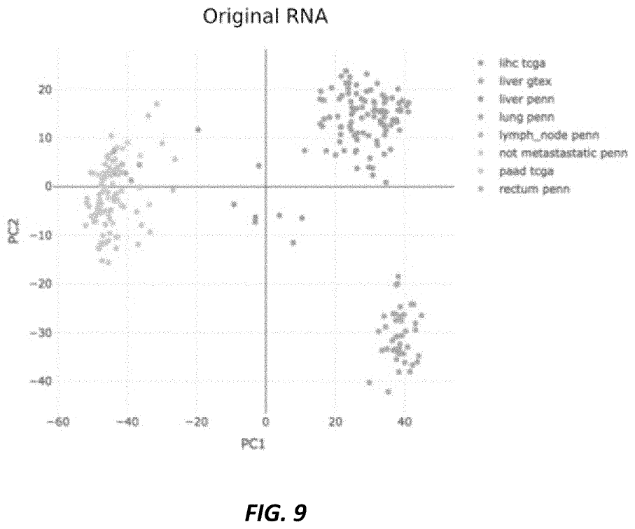

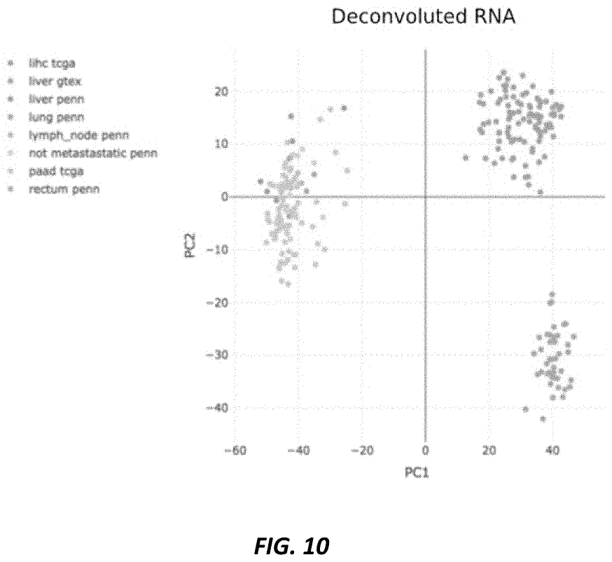

In an example, we validated the liver deconvolution model with a pancreatic cancer research dataset. We identified 65 pancreatic cancer samples from a pancreatic research cohort that included metastatic samples from the liver (9), lung (5), lymph node (1) and rectum (1). Principal component analysis (PCA) of gene expression showed metastatic liver samples (blue) grouped between liver samples (TCGA-teal and GTEx-orange) and all other pancreatic samples (FIG. 9). Metastatic samples from the lung (pink), lymph node (green) and rectum (grey) grouped with PENN (yellow) and TCGA (light brown) primary pancreatic cancers and did not show a large proportion of variation explained by the background tissue site. To adjust for the liver background gene expression, we applied the deconvolution model from the present techniques on the nine liver metastases and showed that the global variation present in the deconvoluted samples grouped together with pancreatic cancer samples (PAAD) as shown in FIG. 10. Thus, as apparent from comparison of the RNA expression data in FIG. 9, pre-deconvolution, versus the deconvoluted expression data of FIG. 10, it is apparent that the liver metastases samples (liver pancreatic metastatic samples in blue) grouped together with samples of known pancreatic cancer after a deconvolution process is performed. A comparison of raw gene expression data to processed gene expression data provided to and/or received from a gene expression analyzer may be used to identify patterns indicating the presence of deconvolution, in some examples.

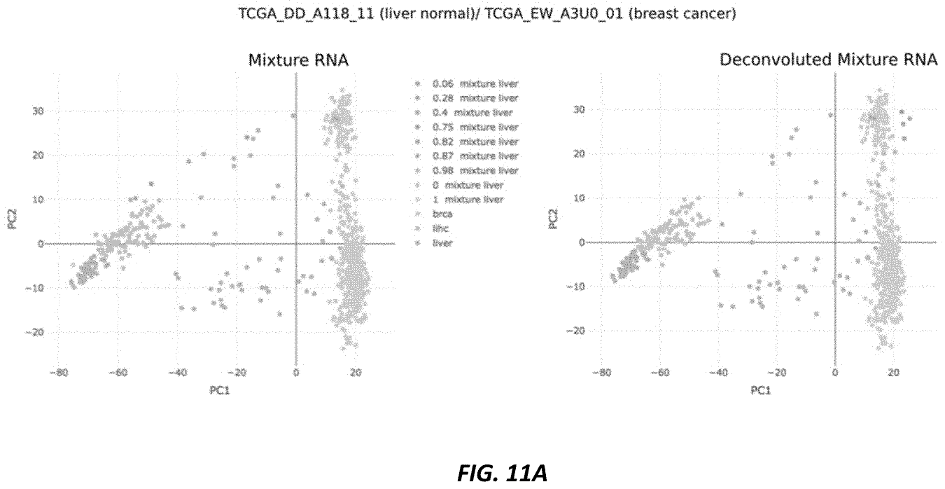

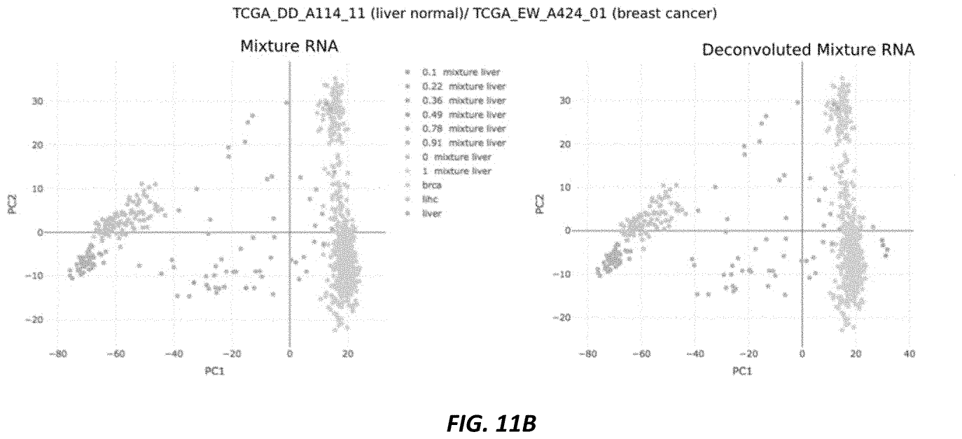

In another example, we validated the liver deconvolution model in silico using breast cancer and normal liver mixtures. To assess the liver deconvolution model with a prior expectation, we performed in silico mixtures of breast cancer and liver normal sequencing reads for two pairs of samples from the TCGA dataset. Specifically, we mixed raw sequence reads for two pairs of samples from TCGA: TCGA_DD_A114_11 (liver normal) with TCGA_EW_A424_01 (breast cancer) and TCGA_DD_A118_11 (liver normal) with TCGA_EW_A3U0_01 (breast cancer). We aligned the sequence reads from each of the four pure, individual samples with a reference sequence, normalized the reads, and selected titration levels at which to combine pairs of samples, based on the number of aligned reads. We created new data files with a combination of the reads from the pairs of samples indicated at five different titration levels, where a titration level is the proportion of the combined reads from the first sample versus the second sample, within the range of 0-100% (see Table 3) for each pair of samples. We used the non-negative least squares (NNLS) model to predict the percentage of liver cluster (the fifth cluster) present in each of the two mixture series (Table 3) followed by deconvolution using a regression model (see, e.g., PCA plots in FIGS. 11A and 11B). The non-negative least squares model accurately approximated the proportion of each mixture that was liver normal reads versus breast cancer reads (Table 3).

As shown in FIGS. 11A and 11B we show that PCA tests performed after deconvolution result in much better grouping of liver samples (right side plots) in comparison to the in silico mixture analysis (left side plots). We found high correlation between the expected proportion of breast cancer reads and the NNLS model predicted tumor percentage (0.89 and 0.82). In addition, the liver deconvolution model performed well at identifying absent liver cell populations in samples with sufficient tumor purity. In sample mixtures with insufficient tumor purity, a tumor percentage over-estimation may result.

Additionally, we examined the performance of expression calls on the deconvoluted samples. We made expression calls, where each call identifies a gene that has a larger (over expression) or smaller (under expression) amount of RNA copies than the gene would have in non-tumorous tissue, where the difference between the amount in the sample and the non-tumorous amount is greater than a user-defined value. The expression calls were made on the pure breast cancer samples and compared the results to the respective mixture and deconvoluted samples.

The first breast cancer sample had MYC gene over expression and PGR and ESR1 under expression. All deconvoluted samples called MYC as overexpressed, while only the 94% breast mixture identified this gene. In this example, only two of the middle range deconvoluted mixtures (82% and 40% liver) identified PGR (progesterone receptor) as under expressed while none of the deconvoluted mixture samples identified ESR1 (estrogen receptor) as underexpressed. The highest liver mixture sample falsely called NGR1 (negative growth regulator protein) as over expressed. Overall, the deconvolution process improved the calling of over expression of MYC across all titrations and decreased false positive calls but was not sensitive enough to capture the two under expressed genes.

The second pure breast cancer sample had PGR and ESR1 over expression. All deconvoluted samples called PGR as over expressed, however, this call was made in all the mixture samples except for the highest proportion of liver. Only the deconvoluted mixture with the lowest liver proportion sample called ESR1 as overexpressed but both of the lowest liver mixtures detected this call. As far as false positives, both of the highest liver deconvoluted mixtures called MYC as overexpressed and the highest liver mixture sample called MTOR as over expressed. In summary, the over expression of PGR in this sample was high enough that its over expression was captured in both analyses. Furthermore, expression calls in samples with low tumor purity, in this particular example, (<22%) was more prone to false positive calls in both the mixture and the deconvoluted sample.

In another example application of the present techniques, we examined expression calls in 124 liver metastatic cancer samples. We selected liver metastatic samples from among four cancers with sample sizes greater than ten, resulting in 124 samples (37 brca, 36 coad, 33 paad and 18 pcpg). We processed each sample through the liver deconvolution model and made expression calls on the original RNA and the deconvoluted RNA sample versus the relevant TCGA cancer and GTEx tissue. For each gene (gene name in the left-most column), we calculated the proportion of samples with that gene called either over or under expressed in i) both RNA datasets, ii) only in the original RNA or iii) only in the deconvoluted RNA (as noted below each column), from among the cancer types where that gene was called at least once. The proportion of samples in each group for which a gene was called over or underexpressed, in each column in FIG. 12, is represented by a shade of pink in a spectrum from pale pink (0, or 0%) to dark purple (0.37, which is 37%).

As shown in FIG. 12, in this example, if none of the samples in a cancer type received an over or under expression call for one of the genes, then all of the samples in that cancer type were excluded from the expression call proportion calculation for that gene. The total number of samples, n, that are included in the sample group for each gene is shown in a column on the right as a shade of green that represents a number in a range of approximately 18 (pale green) to approximately 124 (dark green).

We compared these gene proportions calls and spatially arranged the rows of genes so that the proportions are organized approximately by numeric value to identify trends following deconvolution, as shown in the expression call comparison analysis in FIG. 12. MTOR, ERBB4 and MET were consistently called as over expressed in the original RNA sample (18.5%, 33.9% and 37.1% of the time, respectively) but not in the respective deconvoluted sample. These genes have consistently higher expression in GTEx normal liver compared to the other normal tissue and are subject to inflated gene expression values in the original RNA sample. On the other hand, PGR was called under expressed only in the original RNA 27% of the time because it has much lower expression in liver normal compared to the other normal samples. Following deconvolution, eight genes were called over expressed and two genes under expressed (EGFR and KRAS) in more than 5% of the samples, which is shown in FIG. 12, third column.

With the present techniques, generation of a deconvolution RNA model of various cancer types provides a trained model that can be used to assess and characterize subsequent tissue samples. For example, a method for tissue analysis may include receiving RNA expression data from a sample, analyzing the received RNA expression data against a deconvoluted RNA expression model, serving as a reference RNA expression data, by performing a deconvolution on the received RNA expression data to remove background expression data. The method further may include comparing the deconvoluted received RNA expression data against the reference RNA expression data and determining from that comparison whether the received RNA expression data matches or differs from the reference RNA expression data, e.g., by determining if predetermined groups correlating to particular cancers are present, and from that comparison determining a cancer type or types for the sample.

Although the disclosure above is focused on the identification of different cancer types, it should be understood that the systems and methods described herein may be useful for the determination of a broad range of tissue types in addition to cancer tumors. For instance, tissue samples from any healthy organs, such as brain, muscle, nerve, skin, etc. may contain a mixture of multiple types of cells that have distinct gene expressions. By utilizing the systems and methods described herein, it is possible to analyze the tissue at hand to determine the expression levels of genes for each type of cell from within the tissue sample. For instance, in the case of the brain, neurons, glial cells, astrocytes, oligodendrocytes, and microglia are examples of types of cells found in brain tissue. Using the disclosure provided for herein, clustering on RNA expression data corresponding to a plurality of samples may be performed, where each sample is assigned to at least one of a plurality of clusters. A deconvoluted RNA expression data model for the relevant brain cells may be generated, wherein the data model comprises at least one cluster identified as corresponding to a biological indication of the cells.

In addition to using the disclosure above on healthy tissue samples, it should be understood by those in the art that the disclosure may be used on other cell populations, collections of cells, populations of cells, etc. which may include stem cells, organoids, and the like. Likewise, other tissue samples which are not cancerous but also not healthy (for instance, lung tissue from patients with a history of smoking) may be examined and analyzed using the systems and methods described above.

The methods and systems described above may be utilized in combination with or as part of a digital and laboratory health care platform that is generally targeted to medical care and research. It should be understood that many uses of the methods and systems described above, in combination with such a platform, are possible. One example of such a platform is described in U.S. patent application Ser. No. 16/657,804, titled “Data Based Cancer Research and Treatment Systems and Methods”, and filed Oct. 18, 2019, which is incorporated herein by reference and in its entirety for all purposes.

For example, an implementation of one or more embodiments of the methods and systems as described above may include micro-services constituting a digital and laboratory health care platform supporting deconvolution. Embodiments may include a single micro-service for executing and delivering deconvolution of genomic data or may include a plurality of micro-services each having a particular role which together implement one or more of the embodiments above.

In another example, the deconvolution methods and systems may be executed in one or more micro-services operating on the platform. In another example, one or more of such micro-services may be part of an order management system in the platform that orchestrates the sequence of events needed to conduct deconvolution at the appropriate time and in the appropriate order of events needed to execute genetic sequencing, such as the sequencing of a patient's tumor tissue or normal tissues for precision medicine deliverables to cancer patients. In another example, a bioinformatics micro-service may include one or more sub-micro-services for provisioning and executing various stages of a bioinformatics pipeline. One such stage of a bioinformatics pipeline includes the deconvolution methods and systems described herein. A micro-services based order management system is disclosed, for example, in U.S. Prov. Patent Application No. 62/873,693, titled “Adaptive Order Fulfillment and Tracking Methods and Systems”, filed Jul. 12, 2019, which is incorporated herein by reference and in its entirety for all purposes.

Where the platform includes a genetic analyzer system, the genetic analyzer system may include targeted panels and/or sequencing probes. An example of a targeted panel is disclosed, for example, in U.S. Prov. Patent Application No. 62/902,950, titled “System and Method for Expanding Clinical Options for Cancer Patients using Integrated Genomic Profiling”, and filed Sep. 19, 2019, which is incorporated herein by reference and in its entirety for all purposes. In one example, targeted panels may enable the delivery of next generation sequencing results for deconvolution according to an embodiment, above. An example of the design of next-generation sequencing probes is disclosed, for example, in U.S. Prov. Patent Application No. 62/924,073, titled “Systems and Methods for Next Generation Sequencing Uniform Probe Design”, and filed Oct. 21, 2019, which is incorporated herein by reference and in its entirety for all purposes.

Where the platform includes a bioinformatics pipeline, the methods and systems described above may be utilized after completion or substantial completion of the systems and methods utilized in the bioinformatics pipeline. As one example, the bioinformatics pipeline may receive next-generation genetic sequencing results and return a set of binary files, such as one or more BAM files, reflecting DNA and/or RNA read counts aligned to a reference genome. The methods and systems described above may be utilized, for example, to ingest the DNA and/or RNA read counts and produce deconvoluted DNA and/or RNA data as a result.

When the digital and laboratory health care platform further includes an automated RNA expression caller, RNA expression levels may be adjusted to be expressed as a value relative to a reference expression level, which is often done in order to prepare multiple RNA expression data sets for analysis to avoid artifacts caused when the data sets have differences because they have not been generated by using the same methods, equipment, and/or reagents. An example of an automated RNA expression caller is disclosed, for example, in U.S. Prov. Patent Application No. 62/943,712, titled “Systems and Methods for Automating RNA Expression Calls in a Cancer Prediction Pipeline”, and filed Dec. 4, 2019, which is incorporated herein by reference and in its entirety for all purposes.

The deconvoluted data generated by the systems and methods disclosed herein may then be passed on to other aspects of the platform, such as variant calling, RNA expression calling, or insight engines.

The pipeline may include an automated RNA expression caller. An example of an automated RNA expression caller is disclosed, for example, in U.S. Prov. Patent Application No. 62/943,712, titled “Systems and Methods for Automating RNA Expression Calls in a Cancer Prediction Pipeline”, and filed Dec. 4, 2019, which is incorporated herein by reference and in its entirety for all purposes.

The digital and laboratory health care platform may further include one or more insight engines to deliver further information, characteristics, or determinations related to a disease state that may be based on genetic and/or clinical data associated with a patient and/or specimen. Exemplary insight engines that may receive the deconvoluted information include a tumor of unknown origin engine, a human leukocyte antigen (HLA) loss of homozygosity (LOH) engine, a tumor mutational burden engine, a PD-L1 status engine, a homologous recombination deficiency engine, a cellular pathway activation report engine, an immune infiltration engine, a microsatellite instability engine, a pathogen infection status engine, and so forth. An example tumor of unknown origin engine is disclosed, for example, in U.S. Prov. Patent Application No. 62/855,750, titled “Systems and Methods for Multi-Label Cancer Classification”, and filed May 31, 2019, which is incorporated herein by reference and in its entirety for all purposes. An example of an HLA LOH engine is disclosed, for example, in U.S. Prov. Patent Application No. 62/889,510, titled “Detection of Human Leukocyte Antigen Loss of Heterozygosity”, and filed Aug. 20, 2019, which is incorporated herein by reference and in its entirety for all purposes. An example of a tumor mutational burden engine is disclosed, for example, in U.S. Prov. Patent Application No. 62/804,458, titled “Assessment of Tumor Burden Methodologies for Targeted Panel Sequencing”, and filed Feb. 12, 2019, which is incorporated herein by reference and in its entirety for all purposes. An example of a PD-L1 status engine is disclosed, for example, in U.S. Prov. Patent Application No. 62/854,400, titled “A Pan-Cancer Model to Predict The PD-L1 Status of a Cancer Cell Sample Using RNA Expression Data and Other Patient Data”, and filed May 30, 2019, which is incorporated herein by reference in its entirety for all purposes. An example of a homologous recombination deficiency engine is disclosed, for example, in U.S. Prov. Patent Application No. 62/804,730, titled “An Integrative Machine-Learning Framework to Predict Homologous Recombination Deficiency”, and filed Feb. 12, 2019, which is incorporated herein by reference and in its entirety for all purposes. An example of a cellular pathway activation report engine is disclosed, for example, in U.S. Prov. Patent Application No. 62/888,163, titled “Cellular Pathway Report”, and filed Aug. 16, 2019, which is incorporated herein by reference and in its entirety for all purposes. An example of an immune infiltration engine is disclosed, for example, in U.S. patent application Ser. No. 16/533,676, titled “A Multi-Modal Approach to Predicting Immune Infiltration Based on Integrated RNA Expression and Imaging Features”, and filed Aug. 6, 2019, which is incorporated herein by reference and in its entirety for all purposes. An additional example of an immune infiltration engine is disclosed, for example, in U.S. Patent Application No. 62/804,509, titled “Comprehensive Evaluation of RNA Immune System for the Identification of Patients with an Immunologically Active Tumor Microenvironment”, and filed Feb. 12, 2019, which is incorporated herein by reference and in its entirety for all purposes. An example of an MSI engine is disclosed, for example, in U.S. patent application Ser. No. 16/653,868, titled “Microsatellite Instability Determination System and Related Methods”, and filed Oct. 15, 2019, which is incorporated herein by reference and in its entirety for all purposes. An additional example of an MSI engine is disclosed, for example, in U.S. Prov. Patent Application No. 62/931,600, titled “Systems and Methods for Detecting Microsatellite Instability of a Cancer Using a Liquid Biopsy”, and filed Nov. 6, 2019, which is incorporated herein by reference and in its entirety for all purposes. An additional example of a PD-L1 status engine is disclosed, for example, in U.S. Prov. Patent Application No. 62/824,039, titled “PD-L1 Prediction Using H&E Slide Images”, and filed Mar. 26, 2019, which is incorporated herein by reference and in its entirety for all purposes.

In another example, where the platform includes a report generation engine, the methods and systems described above may be utilized to create a summary report of deconvoluted information for presentation to a physician. For instance, the report may provide to the physician information about the extent to which the specimen that was sequenced contained tumor or normal tissue from a first organ, a second organ, a third organ, and so forth. For example, the report may provide a genetic profile for each of the tissue types, tumors, or organs in the specimen. The genetic profile may represent genetic sequences present in the tissue type, tumor, or organ and may include variants, expression levels, information about gene products, or other information that could be derived from genetic analysis of a tissue, tumor, or organ. The report may include therapies and/or clinical trials matched based on a portion or all of the deconvoluted information. For example, the therapies may be matched according to the systems and methods disclosed in U.S. Prov. Patent Application No. 62/804,724, titled “Therapeutic Suggestion Improvements Gained Through Genomic Biomarker Matching Plus Clinical History”, filed Feb. 12, 2019, which is incorporated herein by reference in its entirety for all purposes. For example, the clinical trials may be matched according to the systems and methods disclosed in U.S. Prov. Patent Application No. 62/855,913, titled “Systems and Methods of Clinical Trial Evaluation”, filed May 31, 2019, which is incorporated herein by reference and in its entirety for all purposes.

The report may include a comparison of the results to a database of results from many specimens. An example of methods and systems for comparing results to a database of results are disclosed in U.S. Prov. Patent Application No. 62/786,739, titled “A Method and Process for Predicting and Analyzing Patient Cohort Response, Progression and Survival”, and filed Dec. 31, 2018, which is incorporated herein by reference and in its entirety for all purposes. The information may be used, sometimes in conjunction with similar information from additional specimens and/or clinical response information, to discover biomarkers or design a clinical trial.

In a third example, the methods and systems described above may be applied to organoids developed in connection with the platform. In this example, the methods and systems may be used to deconvolute genetic sequencing data derived from an organoid to provide information about the extent to which the organoid that was sequenced contained a first cell type, a second cell type, a third cell type, and so forth. For example, the report may provide a genetic profile for each of the cell types in the specimen. The genetic profile may represent genetic sequences present in a given cell type and may include variants, expression levels, information about gene products, or other information that could be derived from genetic analysis of a cell. The report may include therapies matched based on a portion or all of the deconvoluted information. These therapies may be tested on the organoid, derivatives of that organoid, and/or similar organoids to determine an organoid's sensitivity to those therapies. For example, organoids may be cultured and tested according to the systems and methods disclosed in U.S. patent application Ser. No. 16/693,117, titled “Tumor Organoid Culture Compositions, Systems, and Methods”, filed Nov. 22, 2019; U.S. Prov. Patent Application No. 62/924,621, titled “Systems and Methods for Predicting Therapeutic Sensitivity”, filed Oct. 22, 2019; and U.S. Prov. Patent Application No. 62/944,292, titled “Large Scale Phenotypic Organoid Analysis”, filed Dec. 5, 2019, which are incorporated herein by reference and in their entirety for all purposes.

In a fourth example, the systems and methods described above may be utilized in combination with or as part of a medical device or a laboratory developed test that is generally targeted to medical care and research. An example of a laboratory developed test, especially one that is enhanced by artificial intelligence, is disclosed, for example, in U.S. Provisional Patent Application No. 62/924,515, titled “Artificial Intelligence Assisted Precision Medicine Enhancements to Standardized Laboratory Diagnostic Testing”, and filed Oct. 22, 2019, which is incorporated herein by reference and in its entirety for all purposes.

It should be understood that the examples given above are illustrative and do not limit the uses of the systems and methods described herein in combination with a digital and laboratory health care platform.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components or multiple components. These and other variations, modifications, additions, and improvements fall within the scope of the subject matter herein.

Additionally, certain embodiments are described herein as including logic or a number of routines, subroutines, applications, or instructions. These may constitute either software (e.g., code embodied on a machine-readable medium or in a transmission signal) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a microcontroller, field programmable gate array (FPGA) or an application-specific integrated circuit (ASIC)) to perform certain operations. A hardware module may also comprise programmable logic or circuitry (e.g., as encompassed within a processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a processor configured using software, the processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connects the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of the example methods described herein can be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method can be performed by one or more processors or processor-implemented hardware modules. The performance of certain of the operations may be distributed among the one or more processors, not only residing within a single machine, but also deployed across a number of machines. In some example embodiments, the processor or processors may be located in a single location (e.g., within a home environment, an office environment or as a server farm), while in other embodiments the processors may be distributed across a number of locations.

The performance of certain of the operations may be distributed among the one or more processors, not only residing within a single machine, but also deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also includes the plural unless it is obvious that it is meant otherwise.

This detailed description is to be construed as an example only and does not describe every possible embodiment, as describing every possible embodiment would be impractical, if not impossible. One could implement numerous alternative embodiments, using either current technology or technology developed after the filing date of this application.

What is claimed:

1. A computer-implemented method comprising:
performing unsupervised clustering on RNA expression data corresponding to a plurality of samples comprising a first plurality of primary cancer samples and a second plurality of mixed purity metastatic cancer samples, where each sample is assigned to at least one of a plurality of clusters;
generating a deconvoluted RNA expression data model comprising at least one cluster identified as corresponding to biological indication of one or more pathologies;
receiving additional RNA expression data of a sample of tumor tissue;
deconvoluting the additional RNA expression data based in part on the deconvoluted RNA expression data model; and
classifying the sample of tumor tissue as the biological indication of one or more pathologies.

2. The computer-implemented method of claim 1, further comprising:
performing the clustering on the RNA expression data with a grade of membership clustering operation.

3. The computer-implemented method of claim 2, further comprising:
performing the grade of membership clustering operation on the RNA expression data iteratively until the at least one cluster corresponding to the biological indication is identified.

4. The computer-implemented method of claim 1, wherein the generated deconvoluted RNA expression data model comprises a first dimension reflecting a number of samples and a second dimension reflecting a number of genes in the RNA expression data.

5. The computer-implemented method of claim 1, wherein the RNA expression data is raw or normalized RNA expression data.

6. The computer-implemented method of claim 5, wherein the normalized RNA expression data includes RNA expression data from at least one reference gene expression dataset.

7. The computer-implemented method of claim 1, wherein the RNA expression data includes RNA expression data from normal tissue samples, and wherein the at least one cluster corresponds to primary cancer as the biological indication.

8. The computer-implemented method of claim 1, wherein the RNA expression data includes RNA expression data for metastatic samples, and wherein the at least one cluster corresponds to metastatic cancer as the biological indication.

9. The computer-implemented method of claim 1, wherein the biological indication is selected from the group consisting of acute lymphocytic cancer, acute myeloid leukemia, alveolar rhabdomyosarcoma, bone cancer, brain cancer, breast cancer (e.g., triple negative breast cancer), cancer of the anus, anal canal, or anorectum, cancer of the eye, cancer of the intrahepatic bile duct, cancer of the joints, cancer of the head or neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, gastrointestinal cancer (e.g., gastrointestinal carcinoid tumor), glioblastoma, Hodgkin lymphoma, hypopharynx cancer, hematological malignancy, kidney cancer, larynx cancer, liver cancer, lung cancer (e.g., non-small cell lung cancer (NSCLC), small cell lung cancer (SCLC), bronchioloalveolar carcinoma), malignant mesothelioma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, and urinary bladder cancer.

10. The computer-implemented method of claim 1, wherein the sample of tumor tissue is obtained from a tissue site selected from the group consisting of liver tissue, breast tissue, pancreatic tissue, colon tissue, bone marrow, lymph node tissue, skin, kidney tissue, lung tissue, bladder tissue, bone, prostate tissue, ovarian tissue, muscle tissue, intestinal tissue, nerve tissue, testicular tissue, thyroid tissue, brain tissue, fluid samples, and any combination thereof.

11. A computer-implemented method comprising:
receiving RNA expression data for a tissue sample of interest;
comparing the received RNA expression data to a deconvoluted RNA expression model comprising at least one cluster identified as corresponding to biological indication of one or more cell types; and
determining one or more cell types present in the tissue sample of interest based on the comparison.

12. The computer-implemented method of claim 11, wherein the tissue sample of interest is selected from the group consisting of liver tissue, breast tissue, pancreatic tissue, colon tissue, bone marrow, lymph node tissue, skin, kidney tissue, lung tissue, bladder tissue, bone, prostate tissue, ovarian tissue, muscle tissue, intestinal tissue, nerve tissue, testicular tissue, thyroid tissue, brain tissue, fluid samples, and any combination thereof.

13. The computer-implemented method of claim 11, wherein the one or more cell types comprises cell populations, collections of cells, populations of cells, stem cells, and/or organoids.

14. The computer-implemented method of claim 11, wherein the tissue sample is brain tissue and wherein the one or more cell types comprises neurons, glial cells, astrocytes, oligodendrocytes, and/or microglia cells.

15. The computer-implemented method of claim 11, wherein the tissue sample of interest is from cancer tissue.

16. The computer-implemented method of claim 11, wherein the tissue sample of interest is from non-cancerous tissue.

17. The computer-implemented method of claim 11, wherein comparing the received RNA expression data to the deconvoluted RNA expression model comprises deconvoluting the received RNA expression data.

18. A method comprising: receiving RNA expression information of a sample of tumor tissue; generating a deconvolution of the RNA expression information; and determining a biological indication of the tumor tissue based in part on the deconvolution.

19. The method of claim 18 wherein the biological indication is a cancer type.

20. The method of claim 18 wherein the tumor tissue originates from an organ.

21. The method of claim 20 wherein the biological indication of the tumor tissue is a metastatic cancer.

22. The method of claim 18, wherein the step of determining a biological indication of the tumor tissue based in part on the deconvolution comprises: generating enriched gene expressions; and classifying the enriched gene expressions in a biological indication data model.

23. The method of claim 22, wherein generating enriched gene expressions comprises: receiving a percent assignment to each cluster of the plurality of clusters; and scaling the RNA expression information for one or more genes based in part on the corresponding membership associations to each cluster.

24. The method of claim 18, wherein the step of determining a biological indication of the tumor tissue based in part on the deconvolution is performed during deconvolution, wherein the deconvolution is performed with one of a supervised machine learning model and a semi-supervised machine learning model.

25. The method of claim 18, wherein the step of determining a biological indication of the tumor tissue based in part on the deconvolution is performed after deconvolution, wherein the deconvolution is performed with an unsupervised machine learning model.

26. The method of claim 18, wherein receiving RNA expression information of a sample of tumor tissue comprises sequencing the sample of tumor to generate RNA expression information.

27. The method of claim 18, wherein receiving a tumor tissue comprises receiving a tissue sample collected by a tumor biopsy method selected from the group consisting of surgical biopsy, skin biopsy, punch biopsy, prostate biopsy, bone biopsy, bone marrow biopsy, needle biopsy, CT-guided biopsy, ultrasound-guided biopsy, fine needle aspiration, aspiration biopsy, blood collection, and a tumor sample collection method known in the art.
